(19) United States
(12) Patent Application Publication
(10) Pub. No.: US 2024/0390456 A1
(43) Pub. Date: Nov. 28, 2024

(54) COMPOSITIONS AND METHODS FOR MODULATING GLYCEMIC RESPONSE

(71) Applicant: Soquel Co-Pharma, LLC, Boise, ID (US)

(72) Inventor: Eric C. MILLER, Boise, ID (US)

(21) Appl. No.: 18/691,727

(22) PCT Filed: Sep. 16, 2022
(86) PCT No.: PCT/US2022/076582

§ 371 Date: Mar. 13, 2024

Related U.S. Application Data
(60) Provisional application No. 63/245,682, filed on Sep. 17, 2021.

(51) Int. Cl. A61K 38/16; A61K 31/23; A61K 35/20; A61K 36/63; A61K 38/17; A61K 38/39; A61K 45/06; A61P 3/10

(52) U.S. Cl. CPC A61K 38/168; A61K 31/23; A61K 35/20; A61K 36/63; A61K 38/1767; A61K 38/39; A61K 45/06; A61P 3/10

(57) ABSTRACT

The present disclosure relates to therapeutic compositions that may be useful for, among other things, controlling glycemic response, increasing satiety, and improving efficacy, compliance, and/or safety of pharmaceuticals. Methods of use, methods of treatment, and kit that relate to the composition are also included in the disclosure The therapeutic composition comprising fatty acid and an amino acid composition, wherein the therapeutic composition has a total number of calories.

### CROSS-REFERENCE TO RELATED APPLICATIONS

This application claims priority to and the benefit of U.S. Application No. 63/245,682 filed on Sep. 17, 2021, the disclosures of which are hereby incorporated herein by reference in its entirety for all purposes.

### BACKGROUND OF THE INVENTION

Metabolic disorders associated with poor glycemic control, such as type-2 diabetes, remain a global public health issue. Type-2 diabetes, also known as type-2 diabetes mellitus and adult-onset diabetes, is a chronic condition that affects the body's ability to regulate and use glucose, resulting in high blood sugar levels that can negatively affect an individual's health. Many individuals with type-2 diabetes will be treated with multiple drugs, but still fail to hit treatment targets, with most individuals progressing to more serious disease and comorbidities. Noale, M. et al; “Polypharmacy in elderly individuals with type 2 diabetes receiving oral antidiabetic medication,” Acta Diabetologica, 2016, 53, 323-330 and Fonseca, V. A.; “Defining and Characterizing the Progression of Type 2 Diabetes,” Diabetes Care, 2009, 32(2), S151-S156. Thus, despite the myriad of treatment options available, including pharmaceuticals and lifestyle changes (e.g., diet and exercise), many individuals with metabolic disorders associated with poor glycemic control are unable to halt the progression of these disorders and face both acute and chronic health consequences. For example, when left unmanaged or insufficiently managed, type-2 diabetes can cause acute complications such as hypoglycemia and hyperosmolar hyperglycemic nonketotic syndrome, as well as chronic complications that can damage the heart (e.g., cardiovascular disease and heart failure), kidneys (e.g., diabetic nephropathy), eyes (e.g., cataracts and retinopathy), and nerves (e.g., diabetic neuropathy). Moreover, individuals with prediabetes or insulin resistance often progress to developing type-2 diabetes.

Some studies have observed the effect of a preload (a calorie-dense food consumed prior to a meal) of protein and/or dietary fat on blood glucose levels (Wu, T. L.; “A Protein Pre-load Enhances the Glucose Lowering Efficacy of Vildagliptin in Type 2 Diabetes,” Diabetes Care, 2016, 39(4), 511-517 and Sun, L. T.; “Impact of preloading either dairy or soy milk on postprandial glycemia, insulinemia and gastric emptying in healthy adults,” European Journal of Nutrition, 2017, 56, 77-87). The studies found that the use of a preload to prepare the body for consumption of carbohydrates can significantly limit the postprandial blood sugar spike that is often observed in individuals following a meal that is high in carbohydrates. Despite these observations, and existing pharmaceutical and lifestyle interventions, there remains a need for therapies that can help individuals manage metabolic disorders associated with poor glycemic control.

### BRIEF SUMMARY OF THE INVENTION

Therapeutic compositions have been developed to address an unmet medical need for individuals having a metabolic disorder or condition that is associated with poor glycemic control. Specifically, incretin-stimulating compositions are detailed herein. The therapeutic compositions detailed herein may be administered alone or in combination with other pharmaceutical agents. They may be used to attenuate a glycemic response in an individual and treat a metabolic disorder or condition associated with poor glycemic control such as prediabetes, insulin resistance, or type-2 diabetes. The therapeutic compositions provided herein comprise a fatty acid and an amino acid composition. The therapeutic compositions may optionally comprise one or more additional pharmaceutical agents. In some embodiments, the therapeutic composition provided herein comprises (a) a fatty acid, and (b) an amino acid composition, wherein the therapeutic composition has a total number of calories, and wherein (a) and (b) taken together comprise a number of calories accounting for at least 50% of the total number of calories in the therapeutic composition, and wherein the therapeutic composition has a caloric density of at least 100 calories per ounce. In some embodiments, the ratio of the (a) fatty acid to the (b) amino acid composition is between 20:1 and 1:20 by weight. In some embodiments, the ratio is between 20:1 and 1:1 by weight. In some embodiments, the ratio is between 20:1 and 5:1 by weight. In some embodiments, the therapeutic composition has a caloric density of about 100 to about 300 calories per ounce.

In some embodiments, the therapeutic composition comprises one fatty acid. In some embodiments, the therapeutic composition comprises more than one fatty acid. In some embodiments, the therapeutic composition comprises a fatty acid that is an unsaturated fatty acid. In some embodiments, the fatty acid comprises a medium-chain triglyceride. In some embodiments, the fatty acid comprises a vegetable oil, such as olive oil, canola oil, avocado oil, coconut oil, peanut oil, walnut oil, sesame oil, soybean oil, almond oil, flaxseed oil, or sunflower oil. In some embodiments, the fatty acid comprises olive oil, such as extra virgin olive oil. In some embodiments, the therapeutic composition comprises two or more fatty acids. In some embodiments, the fatty acid comprises olive oil and a medium-chain triglyceride oil.

In some embodiments, the amino acid composition comprises a straight-chain amino acid or a branched-chain amino acid. In some embodiments, the amino acid composition comprises whey protein, whey protein isolate, collagen protein, collagen peptide, plant-based protein, insect-based protein, or a fragment of any of the foregoing. In some embodiments, the amino acid composition comprises pea protein, hemp protein or cricket protein, or a fragment of any of the foregoing.

In some embodiments, the therapeutic composition further comprises a microparticle having a size of between about 500 microns and about 3000 microns across at the widest point of the microparticle. In some embodiments, the microparticle comprises the amino acid composition and is suspended in a carrier. In some embodiments, the carrier comprises the fatty acid. For example, in some embodiments, the therapeutic composition comprises olive oil as the fatty acid and a microparticle comprising the amino acid composition, wherein the microparticle is suspended in the olive oil.

In some embodiments, the therapeutic composition further comprises an additional agent. In some embodiments, the therapeutic composition comprises two or more additional agents. In some embodiments, the additional agent is dissolved or suspended in the therapeutic composition. In some embodiments wherein the therapeutic composition comprises a microparticle, the microparticle comprises the additional agent. In some embodiments, the additional agent is a micronutrient, a probiotic, a stimulant, or a pharmaceutical agent. In some embodiments, the additional agent is selected from the group consisting of ergocalciferol, cholecalciferol, calcium, pectin, psyllium, caffeine, metformin, insulin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone. In some embodiments, the additional agent is selected from the group consisting of ergocalciferol, cholecalciferol, calcium, pectin, psyllium, caffeine, metformin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone. Thus, it is understood that in some embodiments, the therapeutic composition is a carrier for an additional agent, such as a drug.

In some embodiments, the therapeutic composition further comprises a flavor-masking agent. In some embodiments, the flavor-masking agent is selected from the group consisting of limonene, methyl salicylate, diacetyl, acetylpropionyl, acetoin, peppermint oil, and cinnamaldehyde. In some embodiments, the flavor-masking agent is cinnamaldehyde.

In some embodiments, the therapeutic composition is packaged as a unit dose. In some embodiments, the unit dose comprises no more than 1.5 ounces of the therapeutic composition. In some embodiments, the unit dose is formulated as a gelatin droplet. In some embodiments, the unit dose is formulated as a liquid-filled capsule or lozenge. In some embodiments, the unit dose is formulated as a suspension packaged in a suitable container. In some embodiments, the suspension contains 40 to 350 calories and has a volume of 0.2 to 1.0 ounces.

In some embodiments, the therapeutic composition is free from a natural or artificial sweetener. In some embodiments, the therapeutic composition further comprises a probiotic. In some embodiments, the probiotic is one or more colonies of microbes. In some embodiments, the therapeutic composition further comprises a fullerene. In some embodiments, the therapeutic composition further comprises a taste receptor agonist with an enteric coating. In some embodiments, the therapeutic composition comprises any two or more of a probiotic, a fullerene, and a taste receptor agonist with an enteric coating.

In some embodiments, the therapeutic composition comprises no more than 5% carbohydrates by weight. In some embodiments, the therapeutic composition comprises no more than 10%, 7%, 3%, or 1% carbohydrates by weight.

In some embodiments, provided herein is a method of stimulating the release of one or more incretins in a subject in need thereof, comprising administering to the subject a therapeutic composition as described herein, wherein the therapeutic composition is administered to the subject not more than one hour prior to energy consumption. In some embodiments, provided herein is a method of attenuating glycemic response in a subject in need thereof, comprising administering to the subject a therapeutic composition as described herein, wherein the therapeutic composition is administered to the subject not more than one hour prior to energy consumption. In some embodiments, provided herein is a method of increasing satiety in a subject in need thereof, comprising administering to the subject a therapeutic composition as described herein, wherein the therapeutic composition is administered to the subject not more than one hour prior to energy consumption.

In some embodiments of the methods described herein, the subject in need thereof has a metabolic disorder or condition associated with poor glycemic control. In some embodiments, the subject in need thereof is at risk of developing a metabolic disorder or condition associated with poor glycemic control. In some embodiments, the subject in need thereof has a metabolic disorder. In some embodiments, the metabolic disorder is prediabetes, insulin resistance, or type-2 diabetes. In some embodiments, the subject is undergoing treatment for a metabolic disorder wherein the treatment comprises a therapy other than the therapeutic composition provided herein. In some embodiments, the subject is concurrently being treated with a pharmaceutical agent selected from the group consisting of metformin, insulin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone.

In some embodiments, provided herein is a method of improving efficacy of a pharmaceutical in a subject, comprising administering to the subject any of the therapeutic compositions as described herein not more than one hour prior to energy consumption, wherein either the subject is concurrently being treating with the pharmaceutical or the therapeutic composition further comprises the pharmaceutical. In some embodiments, the pharmaceutical is a pharmaceutical used to treat a metabolic disorder. In some embodiments, the pharmaceutical is selected from the group consisting of metformin, insulin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone.

In some embodiments of the methods described herein, such as a method of treating a metabolic disorder or condition in a subject in need thereof, the therapeutic composition is administered to the subject not more than 5, 10, 15, 20, 25, 30, 35, 40, 45, 50, 55, or 60 minutes prior to routine energy consumption. In some embodiments of the methods described herein, the therapeutic composition is administered to the subject at about any of 5, 10, 15, 20, 25, 30, 35, 40, 45, 50, 55, or 60 minutes prior to routine energy consumption. In some embodiments, the therapeutic composition is administered to the subject between about 5 and about 60 or between about 5 and about 45 or between about 5 and about 30 or between about 15 and about 60 or between about 15 and about 45 or between about 15 and about 30 or between about 20 and about 40 or between about 30 and about 60 or between about 45 and about 60 minutes prior to routine energy consumption. In some embodiments, the therapeutic composition is administered to the subject about 30 minutes prior to routine energy consumption.

Also provided herein are kits comprising the therapeutic composition as described herein, as well as suitable packaging.

### BRIEF DESCRIPTION OF THE DRAWINGS

FIG. 1 compares and contrasts the effect of the incretins GLP-1 and GIP on physiological processes and responses.

FIG. 2 depicts one embodiment of the invention, wherein microparticles (B) of substantially the same size comprising an amino acid composition are shown suspended in a liquid comprising a fatty acid (A).

FIG. 3A illustrates that microparticles (B) can be of different sizes and can comprise various amino acid compositions and/or additional agents to alter the properties and effects of the therapeutic composition. FIG. 3B illustrates a cross section of one embodiment of a microparticle, wherein the stars represent an amino acid composition and the triangles and circles each represent additional agents.

FIG. 4 is a graph depicting glycemic response of 20 adults at baseline, 15 minutes, 30 minutes, 60 minutes, and 120 minutes after consumption of CHO, FATPRO, and COMBO.

FIG. 5 is the Area Under the Curve (AUC) for whole blood glucose of 20 adults over 120 minutes after CHO consumption, after FATPRO consumption, and after consumption of COMBO.

FIG. 6 is a graph comparing glycemic response of men and women at baseline, 15 minutes, 30 minutes, 60 minutes, and 120 minutes after consumption of CHO, FATPRO, and COMBO.

FIG. 7 is the Area Under the Curve (AUC) comparison of whole blood glucose for men and women over 120 minutes after consumption of CHO, FATPRO, and COMBO.

FIG. 8 is a graph comparing glycemic response of people with “high” and “low” BMIs (Body Mass Index above 25 and less than or equal to 25, respectively) at baseline, 15 minutes, 30 minutes, 60 minutes, and 120 minutes after consumption of CHO, FATPRO, and COMBO.

FIG. 9 is the Area Under the Curve (AUC) comparison of whole blood glucose for people of “high” and “low” BMIs (Body Mass Index above 25 and less than or equal to 25, respectively) over 120 minutes after consumption of CHO, FATPRO, and COMBO.

FIG. 10 is a graph comparing glycemic response of people who took FATPRO with olive oil and those who took FATPRO with MCT oil at baseline, 15 minutes, 30 minutes, 60 minutes, and 120 minutes after consumption of CHO, FATPRO, and COMBO.

FIG. 11 is the Area Under the Curve (AUC) comparison of whole blood glucose for people who took FATPRO with olive oil and those who took FATPRO with MCT oil over 120 minutes after consumption of CHO, FATPRO, and COMBO.

FIG. 12 is a table demonstrating the tunable nature of the therapeutic compositions by adding various excipients to target particular subject populations.

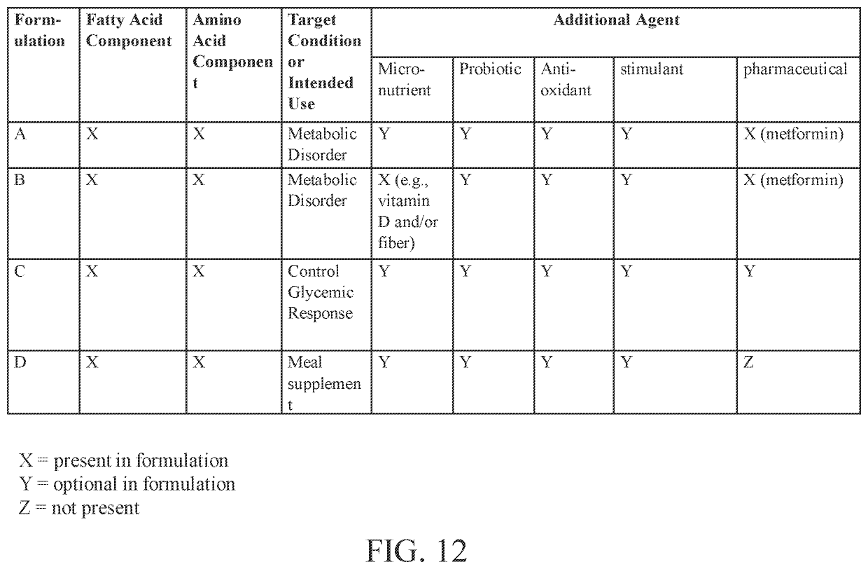

### DETAILED DESCRIPTION OF THE INVENTION

There is an unmet need for therapies that can reliably serve as effective treatment for individuals that currently have or are at risk of developing a metabolic disorder or condition associated with poor glycemic control. The present disclosure is directed to therapeutic compositions comprising a fatty acid and an amino acid composition that address this need. The therapeutic compositions provided herein are defined by reliable and reproducible parameters (e.g., weight and relative ratios of the components), are capable of being tunable to the needs of the individual, can induce the desired physiological response with a minimum possible dose, and can avoid undesirable side effects associated with other therapeutic options. The therapeutic compositions may be suitable for treating a metabolic disorder or condition associated with poor glycemic control such as prediabetes, insulin resistance, or type-2 diabetes, and can be used in conjunction with existing therapies. The therapeutic compositions may be administered to an individual prior to a meal in order to attenuate the individual's glycemic response to routine energy consumption. Acutely, the therapeutic compositions may improve glycemic control and increase satiety. Over time, regular and consistent use of the therapeutic compositions may improve insulin sensitivity in the individual. The therapeutic compositions can be used in conjunction with a pharmaceutical that treats metabolic disorders (e.g., metformin) to improve therapeutic outcomes in individuals taking such medication. The therapeutic compositions may comprise a pharmaceutical such as metformin or may be administered to an individual who is concurrently taking such a pharmaceutical in a separate dosage form.

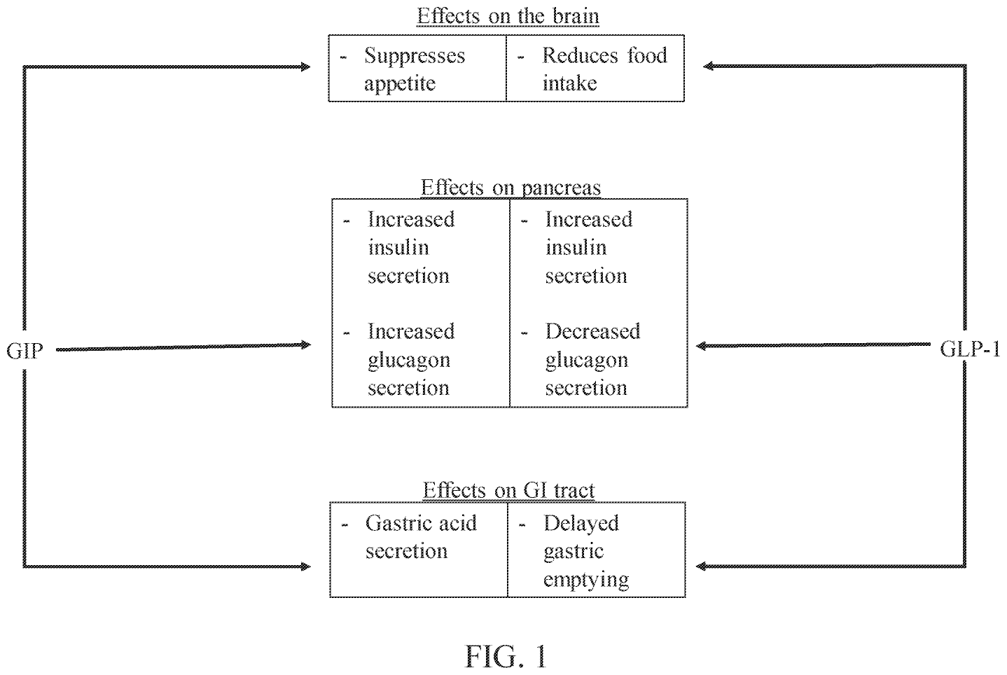

The consumption of amino acids (including proteins) and/or lipids (including triglycerides, e.g., dietary fats) is known to affect several physiological processes via the secretion of the incretin hormones glucagon-like peptide 1 (GLP-1) and glucose-dependent insulinotropic polypeptide (GIP) (Seino, Y. F.; “GIP and GLP-1, the two incretin hormones: Similarities and differences,” Journal of Diabetes Investigation, 2010, 1). GLP-1 and GIP both influence digestion and absorption of nutrients, and although some effects of these two hormones are similar, there are also notable differences in their influence on the body (see FIG. 1). GLP-1 and GIP stimulate the release of insulin from the pancreas and can inhibit hunger signals (Seino, 2010). GLP-1 can also delay gastric emptying, whereas GIP has little to no effect on the rate/timing of gastric emptying (Seino, 2010). GIP stimulates the release of glucagon, which increases the amount of glucose in the blood; conversely, GLP-1 inhibits the release of glucagon (Seino, 2010). The hormones released in the gut, including the incretins, can also influence appetite signaling in the brain. Both GIP and GLP-1 are known to affect regulation of appetite and satiety in the brain and evidence suggests that GIP and GLP-1 interact with the brain to reduce food intake by increasing satiety (Seino, 2010). Because fats and proteins stimulate the release of incretins, the macronutrient composition of meals and the timing of energy consumption can have significant impacts on physiological processes related to metabolism and the uptake of macronutrients.

Without wishing to be bound by theory, the therapeutic compositions provided herein are believed to stimulate the release of incretins and can thus be administered to an individual prior to routine energy consumption in order to attenuate the glycemic response to such energy consumption and/or increase satiety. The therapeutic compositions may provide a consistent and reproducible source of incretin-stimulating components without the liabilities associated with alternative methods of inducing a similar biological response. For example, reducing the number of drugs an individual is taking can reduce the risk of side effects and complications (Noale, M. et al; “Polypharmacy in elderly individuals with type 2 diabetes receiving oral antidiabetic medication,” Acta Diabetologica, 2016, 53, 323-330). Additionally, a therapeutic composition can limit (for example, contain no more than 5 or 3 or 1 percent by weight), or eliminate, the presence in the therapeutic composition of unhealthy fats, carbohydrates, flavoring agents, or other compounds that may reduce the effectiveness of the therapeutic composition. In addition, the present therapeutic compositions may be formulated to contain a relatively large number of calories relative to volume, ensuring that the desired physiological response is achieved with a small volume of therapeutic composition, which can be beneficial for patient compliance. In some embodiments, the therapeutic composition is formulated to elicit a physiological response with a small number of calories and a small volume relative to the number of calories and volume of the following macronutrient consumption. In some embodiments, the number of calories is the minimum number of calories that will reproducibly influence the desired physiological response of an individual. In some embodiments, the therapeutic composition comprises 100-300 calories per fluid ounce, wherein the majority of those calories (e.g., at least 50, 60, 70, 80, 90, 95, or 98 percent of total calories) come from the protein and fat content of the therapeutic composition. The therapeutic compositions can thus be used as a microdose of incretin-stimulating agents that individuals can incorporate into their dietary regimen prior to macronutrient consumption in order to help manage their glycemic response and/or increase satiety and/or improve their sensitivity to insulin. The microdose may be consumed within a suitable time period prior to macronutrient consumption (e.g., within any one of 5 or 10 or 15 or 30 or 60 minutes prior to consuming a meal) such that at least a portion of a physiological process altered by the microdose (e.g., lowering glycemic response and/or increasing satiety) is maintained at the time energy consumption is commenced. In some embodiments, a therapeutic composition comprises 100-300 calories per fluid ounce, wherein the majority of those calories come from the protein and fat content of the therapeutic composition. The therapeutic compositions may further comprise micronutrients such as fiber, vitamins, and/or minerals. The present therapeutic compositions are thus an attractive alternative or complement to existing therapies.

It is understood that embodiments described herein as “comprising” may include “consisting of” and/or “consisting essentially of” aspects and variations.

### Definitions

The term “microdose,” as used herein, refers to a therapeutic composition that is consumed by a subject prior to routine energy consumption. Routine energy consumption refers to any regular process by which a subject ingests calories (e.g. a meal). Following ingestion, the microdose influences physiological processes in the subject (e.g., lowering glycemic response or increasing satiety). A microdose may be consumed within a suitable time period prior to macronutrient consumption (e.g., within any one of 5 or 10 or 15 or 30 or 45 or 60 minutes prior to consuming a meal) such that at least a portion of a physiological process altered by the microdose (e.g., lowering glycemic response and/or increasing satiety) are maintained at the time energy consumption is commenced. A microdose is formulated to elicit a physiological response with a small number of calories and a small volume relative to the number of calories of the following macronutrient consumption. In some embodiments, the number of calories is the minimum number of calories that will reproducibly influence the physiological response of a subject.

The term “therapeutic composition,” as used herein, refers to a composition comprising a fatty acid and an amino acid composition as detailed herein. The therapeutic compositions may be formulated to minimize the number of carbohydrates that can increase glycemic response upon administration to a subject.

The term “fatty acid,” as used herein, includes both free fatty acids and molecules that incorporate fatty acid moieties. The term is inclusive with respect to esters of fatty acids and glycerol, i.e. mono-, di-, and triglycerides. The fatty acid moieties in one di- or triglyceride molecule may be the same or different from each other. The term “fatty acid” is inclusive with respect to mixtures of fatty acids or esters thereof. The term “fatty acid” includes synthetic compounds, such as medium-chain triglyceride (MCT) oil, and naturally occurring compounds, such as vegetable oils (e.g., olive oil) and animal fats. It is understood that embodiments reciting a “fatty acid” include aspects and variations that recite “an ester of a fatty acid” or “a fatty acid or ester thereof” instead of a “fatty acid”.

The term “amino acid composition,” as used herein, includes free amino acids, amino acid derivatives, oligopeptides, polypeptides, proteins, and any combination thereof, including fragments thereof. There may be multiple free amino acids, multiple amino acid derivatives, multiple oligopeptides, multiple polypeptides, and/or multiple proteins in the amino acid composition, including fragments thereof. Amino acid derivatives include, without limitation, amino acid esters, hydroxy amino acids, and amino acid halides. The term “amino acid” includes both free proteinogenic and non-proteinogenic amino acids and polypeptides, oligopeptides, polypeptides, and proteins thereof, including fragments of any of the foregoing.

The term “microparticle,” as used herein, refers to a particle having a size between 1 μm and 5 mm across at its widest point that is suitable for consumption. The microparticle comprises a coating material containing an active material. The term includes, without limitation, microspheres, microcapsules, and microbeads, such that all possible shapes of particle are included by the term “microparticle(s)”. The term “microparticle” is inclusive with respect to a collection of microparticles, which may contain one or more types of microparticle. It is understood that when a range of sizes for the microparticle is recited, embodiments wherein the microparticle is a collection of microparticles with a size distribution falling within the recited range are also contemplated.

As used herein, the term “unit dose” or “unit dosage” or “unit” refers to a physically discrete unit that contains a predetermined quantity of therapeutic composition calculated to produce a desired therapeutic effect. The unit dose or unit dosage or unit may be in the form of a tablet, capsule, sachet, etc. referred to herein as a “unit dosage form”.

The term “body mass index,” or “BMI,” as used herein, is defined as the body mass divided by the square of the body height, and is expressed in units of kg/m2, resulting from mass in kilograms and height in meters.

Reference to “about” a value or parameter herein includes (and describes) variations that are directed to that value or parameter per se. For example, descriptions referring to “about X” include descriptions of “X” per se and descriptions referring to from “about X” to “about Y” include descriptions of from “X” to “Y” per se.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. As it relates to a metabolic disorder, examples of the beneficial or desired results include, without limitation, attenuation of glycemic response, reduced reliance on pharmaceuticals, reduced disease progression, and stimulation of incretins.

The disclosures of all publications, patents, and patent applications referred to herein are each hereby incorporated by reference in their entireties.

### Compositions

Therapeutic compositions are provided herein wherein the therapeutic composition comprises a component that is a fatty acid and a component that is an amino acid composition. In some embodiments, the amino acid component is suspended in the fatty acid component. In some embodiments, the therapeutic compositions optionally comprise one or more of a flavor-masking agent, pharmaceutical, food additive, macronutrient, micronutrient, natural product, microorganism, cell, catalyst, or any combination thereof.

The therapeutic compositions disclosed herein may be calorie-dense with respect to the number of calories per unit volume. In some embodiments, the therapeutic composition contains at least 100, at least 150, at least 200, at least 250, or at least 300 calories per ounce. In some embodiments, the therapeutic composition contains no more than 350 calories per ounce. In some embodiments, the therapeutic composition contains about 100 to about 150, about 200, about 250, or about 300 calories per ounce. In some embodiments, the therapeutic composition contains about 150 to about 200, about 250 or about 300 calories per ounce. In some embodiments, the therapeutic composition contains about 200 to about 250 or about 300 calories per ounce. In some embodiments, the therapeutic composition contains about 250 to about 300 calories per ounce. In some embodiments, the fatty acid and amino acid composition of the therapeutic composition together account for at least 50%, at least 60%, at least 70%, at least 80%, at least 90%, at least 95%, at least 96%, at least 97%, at least 98%, or at least 99% of the total calories in the therapeutic composition.

The therapeutic compositions comprise a fatty acid and an amino acid composition. In some embodiments, the ratio of the fatty acid to amino acid composition is between about 50:1 and about 1:50, between about 20:1 and about 1:20, between about 10:1 and about 1:10, between about 5:1 and about 1:5, between about 20:1 and about 1:1, between about 10:1 and about 1:1, or between about 5:1 and about 1:1 by weight. In some embodiments, the ratio of the fatty acid to the amino acid composition is at least 1:2, at least 1:1, or at least 5:1 by weight. In some embodiments, the ratio of the fatty acid to the amino acid composition is no greater than 100:1, 50:1, 30:1, 20:1, or 10:1 by weight.

In some embodiments, the fatty acid is an ester of one or more fatty acids and glycerol. In some embodiments, the fatty acid is a triglyceride. In some embodiments, the fatty acid is a long-chain fatty acid. In some embodiments, the fatty acid is a medium-chain fatty acid. In some embodiments, the fatty acid is a synthetic oil. In some embodiments, the fatty acid is medium-chain triglyceride (MCT) oil. In some embodiments, the fatty acid is a vegetable oil. In some embodiments, the vegetable oil is olive oil, canola oil, avocado oil, coconut oil, peanut oil, walnut oil, sesame oil, soybean oil, almond oil, flaxseed oil, or sunflower oil. In some embodiments, the fatty acid is olive oil. In some embodiments, the fatty acid is coconut oil. In some embodiments, the fatty acid is almond oil. In some embodiments, the fatty acid is virgin olive oil. In some embodiments, the fatty acid is extra virgin olive oil. In some embodiments, the olive oil used in the therapeutic composition is prepared using particular processing techniques. In some embodiments, the olive oil in the therapeutic composition was made from olives subjected to destoning. In some embodiments, the olive oil in the therapeutic composition was subjected to a malaxation process carried out in a hermetic malaxer. In some embodiments, the fatty acid is an omega-3 fatty acid. In some embodiments, the fatty acid comprises one or more monounsaturated fatty acids. In some embodiments, the one or more monounsaturated fatty acids account for 25% or more, 50% or more, or 75% or more of the fatty acid in the composition.

In some embodiments, the fatty acid is a combination of two or more fatty acids. In some embodiments, the fatty acid comprises one or more fatty acids selected from the group consisting of vegetable oil, synthetic oil, long chain fatty acids, and medium-chain fatty acids. In some embodiments, the fatty acid comprises two or more fatty acids selected from the group consisting of olive oil, canola oil, avocado oil, coconut oil, peanut oil, walnut oil, sesame oil, soybean oil, almond oil, flaxseed oil, sunflower oil, and medium-chain triglyceride (MCT) oil. In some embodiments, the fatty acid contains two or more of long-chain fatty acids, medium-chain fatty acids, omega-3 fatty acids, and monounsaturated fatty acids. In some embodiments, the fatty acid comprises two or more of any one of the fatty acids listed herein as though each combination were specifically and individually listed.

The therapeutic composition further comprises an amino acid composition. In some embodiments, the amino acid composition is selected from the group consisting of a straight-chain amino acid and a branched-chain amino acid. In some embodiments, the amino acid composition is selected from the group consisting of whey protein, whey protein isolate, collagen protein, collagen peptide, plant-based protein, and insect-based protein. In some embodiments, the amino acid composition comprises two or more amino acids selected from the group consisting of a straight-chain amino acid, a branched-chain amino acid, whey protein, whey protein isolate, collagen protein, plant-based protein, insect-based protein, and fish-based protein. In some embodiments, the plant-based protein comprises pea protein and/or hemp protein. In some embodiments, the insect-based protein comprises cricket protein. In some embodiments, the fish-based protein comprises sardine protein.

In some embodiments, the amino acid composition comprises two or more amino acids selected from the group consisting of a straight-chain amino acid, a branched-chain amino acid, whey protein, whey protein isolate, collagen protein, collagen peptide, plant-based protein, and insect-based protein. In some embodiments, the amino acid composition contains one or more amino acids selected from the group consisting of a straight-chain amino acid, a branched-chain amino acid, whey protein, whey protein isolate, collagen protein, collagen peptide, plant-based protein, insect-based protein, and fish-based protein. In some embodiments, the amino acid composition contains one or more amino acids selected from the group consisting of pea protein, hemp protein, cricket protein, and fish protein. In some embodiments, the amino acid composition is a complete protein (contains the 9 essential amino acids). In some embodiments, the amino acid composition is a single protein source (e.g., whey protein). In some embodiments, the amino acid composition is from one or more plant sources. In some embodiments, the amino acid composition comprises two or more of the amino acids listed herein as though each combination of amino acids were specifically and individually listed.

In some embodiments, the composition further comprises a microparticle. In some embodiments, the microparticle comprises the amino acid composition. In some embodiments, the amino acid composition is located within the microparticle or within a collection of microparticles.

In some embodiments, the microparticle comprises a coating. In some embodiments, the coating comprises a polymer. In some embodiments, the polymer is of natural origin. In some embodiments, the polymer is of synthetic origin. In some embodiments, the polymer is hydrophobic. In some embodiments, the polymer is hydrophilic. In some embodiments, the coating is a lipid. In some embodiments, the microparticle comprises a collection of microparticles. The microparticles comprising the collection of microparticles may have the same or different coatings, the same or different amino acid compositions, and the same or different additional agents within the microparticles. In some embodiments, the microparticle has a diameter or a range of diameters between 500 and 3000 microns. In some embodiments, the microparticle has a diameter or a range of diameters between 750 and 2500 microns. In some embodiments, the microparticle has a diameter or a range of diameters between 1000 and 2000 microns. In some embodiments, the microparticle has a diameter or a range of diameters below 3000, below 2500, below 2000, below 1500, or below 1000. In some embodiments, the microparticle has a diameter above 500 microns, above 1000 microns, above 1500 microns, or above 2000 microns. In some embodiments, the microparticle does not comprise microparticles larger than 2500 microns, larger than 2000 microns, larger than 1500 microns, or larger than 1000 microns in diameter. In some embodiments, the microparticle does not comprise microparticles smaller than 750 or smaller than 500 microns in diameter.

In some embodiments, the microparticle comprises carbohydrates. In some embodiments, the carbohydrates in the microparticle do not induce a blood sugar response. In some embodiments, the carbohydrates in the microparticle induce a negligible blood sugar response. In some embodiments, the carbohydrates in the coating induce a negligible increase in blood sugar. In some embodiments, the microparticles account for less than a 10% increase in blood sugar levels compared to baseline blood sugar levels. In some embodiments, the microparticles account for less than a 5% increase in blood sugar levels compared to baseline blood sugar levels. In some embodiments, the microparticles account for less than a 2% increase in blood sugar levels compared to baseline blood sugar levels.

In some embodiments, the therapeutic composition further comprises a flavor-masking agent. In some embodiments, the flavor-masking agent is a natural flavoring substance. In some embodiments, the flavor-masking agent is an artificial flavoring substance. In some embodiments, the flavor-masking agent is cinnamon oil. In some embodiments, the flavor-masking agent is cinnamaldehyde. In some embodiments, the flavor-masking agent is selected from the group consisting of limonene, methyl salicylate, diacetyl, acetylpropionyl, acetoin, peppermint oil, and cinnamaldehyde. In some embodiments, the flavor-masking agent contains a combination of natural flavoring substances and/or artificial flavoring substances. In some embodiments, the flavor-masking agent comprises two or more compounds selected from the group consisting of limonene, methyl salicylate, diacetyl, acetylpropionyl, acetoin, peppermint oil, and cinnamaldehyde. In some embodiments, the therapeutic composition is flavor neutral. In some embodiments, the therapeutic composition does not comprise a sweetener. In some embodiments, the therapeutic composition does not contain a saccharide. In some embodiments, the therapeutic composition does not comprise a flavor-masking agent. In some embodiments, the flavor-masking agent does not induce a glycemic response when ingested.

In some embodiments, the therapeutic composition mimics the protein and fat content of a particular food and comprises at least one biologically-active compound with health benefits present in the food. In some embodiments, the particular food is a fish. In some embodiments, the fish is a sardine. In some embodiments, the particular food is an egg. In some embodiments, the therapeutic composition has a number of calories sufficient to be a meal replacement. In some embodiments, the therapeutic composition is a microdose of protein and fat present in the particular food.

In some embodiments, the therapeutic composition has a low percentage of carbohydrates, such as carbohydrates known to elicit a robust glycemic response (e.g., sugar and starch). In some embodiments, the therapeutic composition comprises less than 10%, less than 5%, less than 3%, less than 1%, or less than 0.5% carbohydrates (e.g., those known to elicit a robust glycemic response) by weight. In some embodiments, the therapeutic composition does not comprise an artificial sweetener. In some embodiments, the therapeutic composition does not comprise an artificial or natural sweetener. In some embodiments, the therapeutic composition comprises carbohydrates that are known to elicit a low glycemic response (e.g., fiber). In some embodiments, the therapeutic composition does not comprise carbohydrates known to elicit a robust glycemic response. In some embodiments, blood sugar levels do not rise more than about 10%, 5%, 2%, or 1% in response to consumption of the therapeutic composition.

In some embodiments, the therapeutic composition is a microdose that is consumed prior to energy consumption. In some embodiments, the energy consumption is a meal. In some embodiments, the therapeutic composition comprises a microparticle suspended in a liquid carrier. In some embodiments, the liquid carrier comprises a fatty acid. In some embodiments, the liquid carrier comprises olive oil. In some embodiments, the liquid carrier comprises medium-chain triglyceride (MCT) oil.

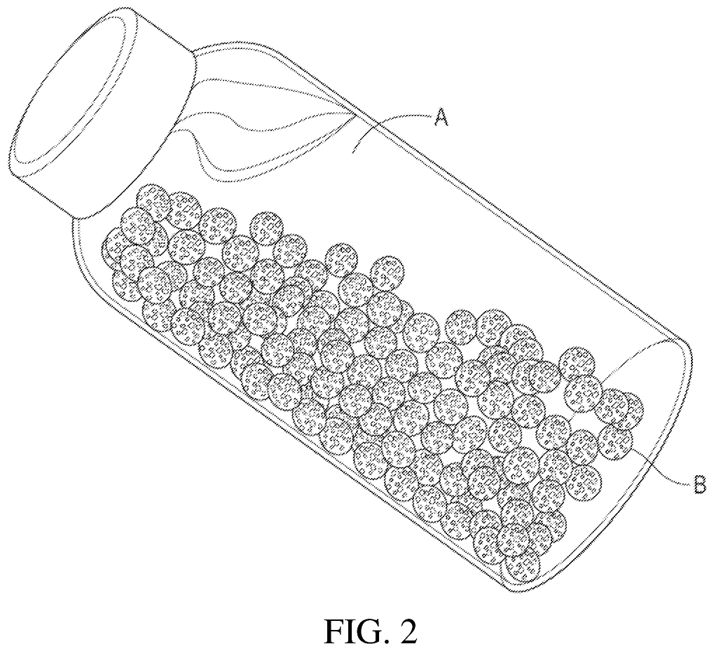

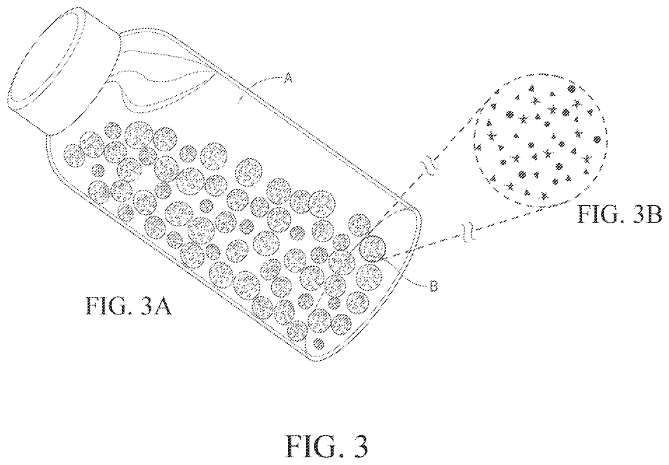

In some embodiments, the therapeutic composition is packaged as a unit dose. In some embodiments, the unit dose is a gelatin droplet, liquid-filled capsule, or lozenge. In some embodiments, the unit dose comprises an enteric coating. In some embodiments, the therapeutic composition is a metered dose and administered or consumed with the aid of a delivery device. In some embodiments, the unit dose is a suspension. In some embodiments, the suspension is contained in a suitable container. In some embodiments, the suitable container is a vial (see FIG. 2 and FIG. 3).

In some embodiments, the unit dose contains at least 40, at least 70, at least 100, at least 150, at least 200, at least 250, at least 300, or at least 350 calories. In some embodiments, the unit dose contains no more than 400 calories. In some embodiments, the unit dose contains about 40 to about 70, about 100, about 150, about 200, about 250, about 300, about 350, or about 400 calories. In some embodiments, the unit dose contains about 70 to about 100, about 150, about 200, about 250, about 300, about 350, or about 400 calories. In some embodiments, the unit dose contains about 100 to about 150, about 200, about 250, about 300, about 350, or about 400 calories. In some embodiments, the unit dose contains about 150 to about 200, about 250, about 300, about 350, or about 400 calories. In some embodiments, the unit dose contains about 200 to about 250, about 300, about 350, or about 400 calories. In some embodiments, the unit dose contains about 250 to about 300, about 350, or about 400 calories. In some embodiments, the unit dose contains about 300 to about 350 or about 400 calories.

In some embodiments, the unit dose has a volume that is smaller than 2.0, smaller than 1.8, smaller than 1.6, smaller than 1.4, smaller than 1.2, smaller than 1.0, smaller than 0.8, smaller than 0.6, or smaller than 0.4 fluid ounces. In some embodiments, the unit dose has a volume that is no smaller than 0.1 fluid ounces. In some embodiments, the unit dose has a volume that is between about 0.2 and about 0.4, about 0.6, about 0.8, about 1.0, about 1.2, about 1.4, about 1.6, about 1.8, or about 2.0 fluid ounces. In some embodiments, the unit dose has a volume that is between about 0.4 and about 0.6, about 0.8, about 1.0, about 1.2, about 1.4, about 1.6, about 1.8, or about 2.0 fluid ounces. In some embodiments, the unit dose has a volume that is between about 0.6 and about 0.8, about 1.0, about 1.2, about 1.4, about 1.6, about 1.8, or about 2.0 fluid ounces. In some embodiments, the unit dose has a volume that is between about 0.8 and about 1.0, about 1.2, about 1.4, about 1.6, about 1.8, or about 2.0 fluid ounces. In some embodiments, the unit dose has a volume that is between about 1.0 and about 1.2, about 1.4, about 1.6, about 1.8, or about 2.0 fluid ounces. In some embodiments, the unit dose has a volume that is between about 1.2 and about 1.4, about 1.6, about 1.8, or about 2.0 fluid ounces. In some embodiments, the unit dose has a volume that is between about 1.4 and about 1.6, about 1.8, or about 2.0 fluid ounces. In some embodiments, the unit dose has a volume that is between about 1.6 and about 1.8 or about 2.0 fluid ounces. In some embodiments, the unit dose has a volume that is between about 1.8 and about 2.0 fluid ounces.

The therapeutic compositions may contain any of the calories or ranges of calories recited above in combination with any of the volumes or ranges of volumes recited above as though each combination were individually and specifically listed. Any of the unit dosage forms, including the unit dosage forms having a particular caloric profile as detailed above, may in some embodiments be present in a dosage form in accordance with the volume parameters detailed herein. For example, in one embodiment, the therapeutic composition or unit dose contains at least 40 calories and has a volume smaller than 2.0 fluid ounces. In another embodiment, the therapeutic composition or unit dose contains about 40 to about 400 calories and has a volume of about 0.2 to about 2.0 fluid ounces.

In some embodiments, the therapeutic composition is useful as a microdose. In some embodiments, the microdose contains about 40, about 70, or about 100 calories and has a volume that is less than 1 fluid ounce, less than 0.8 fluid ounces, less than 0.6 fluid ounces, or less than 0.4 fluid ounces. In some embodiments, the therapeutic composition is useful as a meal replacement. In some embodiments, the meal replacement contains at least 150, at least 200, at least 250, or at least 300 calories and the volume is less than about 1 fluid ounce, less than about 1.2 fluid ounces, less than about 1.4 fluid ounces, less than about 1.6 fluid ounces, less than about 1.8 fluid ounces, or less than about 2.0 fluid ounces.

In some embodiments, the therapeutic composition comprises compounds that are associated with at least one health benefit. In some embodiments, the health benefit is increased cardiovascular health and/or decreased cardiovascular risk. In some embodiments, the health benefit is an improvement to insulin sensitivity. In some embodiments, the compounds have antioxidant, anti-inflammatory, antihypertensive, neuroprotective, antibacterial, chemotherapeutic, or immune-modulatory effects. In some embodiments, the compounds are known to prevent adverse gastrointestinal effects such as ulcerative colitis, Crohn's disease, gastric cancer, and peptic ulcers. In some embodiments, the compounds are biophenols. Examples of biophenols include hydroxytyrosol, tyrosol, oleocanthal, oleacin, oleuropein, and verbascoside. In some embodiments, the compounds are present in the source of the fatty acid. In some embodiments, the fatty acid is olive oil. In some embodiments, the olive oil is virgin olive oil. In some embodiments, the olive oil is extra virgin olive oil. See Riccardi, G., et al.; “Dietary Far, Insulin Sensitivity and Metabolic Syndrome,” Clin. Nutr., 2004, 23, 447-456 and Boskou, D.; “Olive and Olive Oil Bioactive Constituents,” 2015, pages 17-18 (both incorporated by reference in their entireties).

In some embodiments, the therapeutic composition comprises biologically active-compounds that are Generally Recognized As Safe (GRAS) by the U.S. Food and Drug Administration. In some embodiments, the biologically-active compounds comprise a fatty acid and an amino acid composition.

### Additional Agents

The therapeutic compositions herein are tunable to the needs of individuals that may, for example and without limitation, have differing dietary needs or have more or less progressed metabolic disorders compared to other individuals. In addition to adjusting the amount and/or type of fat and protein content of the therapeutic compositions, the therapeutic compositions may also contain additional agents that are selected based on the individual's needs. The additional agent may be a pharmaceutical, micronutrient, or other compound that would improve the health of the individual.

In some embodiments, the therapeutic composition further comprises an additional active agent. In some embodiments, the additional active agent is dissolved or suspended in the fatty acid. In some embodiments, the microparticle comprises the additional active agent. In some embodiments, an additional active agent is dissolved or suspended in the fatty acid and the microparticle comprises an additional active agent (which may be the same or different additional active agent as that dissolved or suspended in the fatty acid). In some embodiments, the therapeutic composition comprises a microparticle that comprises the amino acid composition and microcapsules that comprise the additional active agent. The active agent may be selected from various groups including, without limitation, pharmaceutically active molecules, food additives, macronutrients (e.g., proteins, carbohydrates, and fats), micronutrients (e.g. vitamins and minerals), natural products (e.g., products derived from plants), microorganisms, cells, catalysts of chemical reactions (including enzymes), or any combination thereof. In some embodiments, the additional active agent is biologically-active.

In some embodiments, the additional agent is a micronutrient. In some embodiments, the micronutrient is known to have a specific benefit related to a particular individual population. In some embodiments, the additional agent is a vitamin. In some embodiments, the additional agent is a mineral. In some embodiments, the additional agent is vitamin D. In some embodiments, the additional agent is ergocalciferol. In some embodiments, the additional agent is cholecalciferol. In some embodiments, the additional agent is a dietary calcium. In some embodiments, the additional agent is Ca²⁺. In some embodiments, the additional agent is a fiber. In some embodiments, the additional agent is pectin. In some embodiments, the additional agent is psyllium.

In some embodiments, the additional agent is a probiotic. In some embodiments, the probiotic comprises one or more colonies of microbes. In some embodiments, the probiotic comprises one or more polysaccharides. In some embodiments, the probiotic comprises one or more glycans.

In some embodiments, the additional agent is an antioxidant. In some embodiments, the antioxidant is a fullerene. In some embodiments, the antioxidant is epigallocatechin gallate.

In some embodiments, the additional agent comprises a taste receptor agonist. In some embodiments, the taste receptor agonist has an enteric coating. Compositions with enteric coatings and taste receptor agonists include, without limitation, those described in WO 2014/074749 and WO 2019/165309, which are incorporated herein by reference in their entireties.

In some embodiments, the additional agent is a stimulant. In some embodiments, the stimulant is caffeine.

In some embodiments, the additional agent is a pharmaceutical that is a medication for type-2 diabetes. Classes of pharmaceuticals used to treat type-2 diabetes include, without limitation, alpha-glucosidase inhibitors, biguanides, dopamine agonists, dipeptidyl peptidase-4 (DPP-4) inhibitors, incretin mimetics, meglitinides, sodium-glucose transporter (SGLT)2 inhibitors, sulfonylureas, and thiazolidinediones. In some embodiments, the additional agent is selected from the group consisting of metformin, insulin, acarbose, miglitol, alogliptin, canagliflozin, dapagliflozin, empagliflozin, bromocriptine, linagliptin, albiglutide, dulaglutide, exenatide, nateglinide, repaglinide, glimepiride, gliclazide, rosiglitazone, and pioglitazone. Incretin mimetics include glucagon-like peptide-1 (GLP-1) receptor agonists and glucose-dependent insulinotropic polypeptide (GIP) receptor agonists.

In some embodiments, the additional agent is a pharmaceutical that is a medication for hypertension. Classes of pharmaceuticals used to treat hypertension include, without limitation, diuretics, beta-blockers, ace inhibitors, angiotensin II receptor blockers, calcium channel blockers, alpha blockers, alpha-2 receptor agonists, central agonists, peripheral adrenergic inhibitors, and vasodilators. In some embodiments, the additional agent is selected from the group consisting of furosemide, bumetanide, atenolol, propranolol, metoprolol, benazepril, lisinopril, irbesartan, losartan, nifedipine, verapamil, doxazosin, prazosin, clonidine, tizanidine, alpha methyldopa, reserpine, guanadrel, and captopril.

In some embodiments, the additional agent is a pharmaceutical or medical food that is used to treat hyperlipidemia and elevated triglycerides. Classes of pharmaceuticals used to treat hyperlipidemia and elevated triglycerides include, without limitation, statins, fibrates, fatty acids, and nicotinic acids. In some embodiments, the additional agent is selected from the group consisting of atorvastatin, fluvastatin, lovastatin, pitavastatin, pravastatin, rosuvastatin, simvastatin, fenoglide, lofibra, lipofen, tricor, lovaza, vascepa, niaspan, and vayarol.

It is understood that for embodiments wherein the active agent is a pharmaceutical, the therapeutic composition comprises a dietary component (e.g., at least the fatty acid component and the amino acid component) that complement the effects of the pharmaceutical. In some embodiments wherein the additional active agent is a pharmaceutical, the pharmaceutical weighs at least 1 mg, at least 5 mg, at least 10 mg, at least 100 mg, or at least 200 mg.

In some embodiments, the additional active agent comprises two or more of the additional active agents listed above as though each combination of additional active agents were specifically and individually listed.

### Methods of Use

The therapeutic compositions detailed herein may be used in the methods provided below. In some embodiments, the therapeutic composition is a microdose, such that the therapeutic composition comprises a component that is a fatty acid and a component that is a an amino acid composition and wherein the therapeutic composition is configured for consumption prior to further energy consumption (such as a unit dosage form comprising about 40, about 70, or about 100 calories and has a volume that is less than about 1 fluid ounce, less than about 0.8 fluid ounces, less than about 0.6 fluid ounces, or less than about 0.4 fluid ounces). In some embodiments, administering the therapeutic composition does not trigger a blood sugar increase in a subject. In some embodiments, the therapeutic composition is administered in a dose that is determined using the subject's BMI. In some embodiments, the subject has a BMI >25, a BMI ≥25, a BMI ≥30, or a BMI ≥30. In some embodiments, the subject is a man. In some embodiments, the subject is a woman.

In some embodiments, the microdose is consumed about 5 minutes, about 10 minutes, about 15 minutes, about 20 minutes, about 30 minutes, about 45 minutes, or about 60 minutes before energy consumption. In some embodiments, the microdose is administered from about 5 to about 10, about 15, about 20, about 30, about 45, or about 60 minutes before energy consumption. In some embodiments, the microdose is consumed from about 10 to about 15, about 20, about 30, about 45, or about 60 minutes prior to energy consumption. In some embodiments, the microdose is consumed from about 15 to about 20, about 30, about 45, or about 60 minutes prior to energy consumption. In some embodiments, the microdose is consumed from about 20 to about 30, about 45, or about 60 minutes prior to energy consumption. In some embodiments, the microdose is consumed from about 30 to about 45 or about 60 minutes prior to energy consumption. In some embodiments, the microdose is consumed from about 45 to about 60 minutes prior to energy consumption.

In some embodiments, a method for controlling glycemic response in a subject is provided, the method comprising administering to a subject in need thereof a therapeutic composition as described above. In some embodiments, a method for lowering glycemic response in a subject is provided, the method comprising administering to a subject in need thereof a therapeutic composition as described above. In some embodiments, a method for attenuating glycemic response is provided, the method comprising administering to a subject in need thereof a therapeutic composition as described above. In some embodiments, a method for preventing hyperglycemia is provided, the method comprising administering to a subject in need thereof a therapeutic composition as described above. In some embodiments, the subject is obese. In some embodiments, the subject is prediabetic. In some embodiments, the subject is insulin resistant. In some embodiments, the subject has type-2 diabetes. In some embodiments, the subject has a disease or condition selected from the group consisting of obesity, prediabetes, insulin resistance, or type-2 diabetes. In some embodiments, the subject has diabetes and is vitamin D deficient. In some embodiments, the subject has type-2 diabetes and is obese. In some embodiments, the subject is undergoing treatment for a metabolic disorder. In some embodiments, the treatment comprises administration of metformin. In some embodiments, the treatment comprises insulin.

The method of controlling, lowering, or attenuating glycemic response lowers the change in peak blood sugar in a subject using the method relative to the change in peak if they were not using the method following an identical energy consumption. In some embodiments, the energy consumption is ingestion of a meal. In some embodiments, the energy consumption is ingestion of a glucose tolerance test beverage. In some embodiments, the average change in peak blood sugar in the subject is lowered by about 10, about 20, about 30, about 40, or about 50 percent. In some embodiments, the average change in peak blood sugar in the subject is about 10 to about 20, about 30, about 40, or about 50 percent. In some embodiments, the average change in peak blood sugar in the subject is between about 20 to about 30, about 40, or about 50 percent. In some embodiments, the average change in peak blood sugar in the subject is between about 30 to about 40 or about 50 percent. In some embodiments, the average change in peak blood sugar in the subject is lowered by about 5, about 10, about 15, about 20, about 25, or about 30 mg/dL. In some embodiments, the average change in peak blood sugar in the subject is lowered by about 5 to about 10, about 15, about 20, about 25, or about 30 mg/dL. In some embodiments, the average change in peak blood sugar in the subject is lowered by about 10 to about 15, about 20, about 25, or about 30 mg/dL. In some embodiments, the average change in peak blood sugar in the subject is lowered by about 15 to about 20, about 25, or about 40 mg/dL. In some embodiments, the average change in peak blood sugar in the subject is lowered by about 20 to about 25 or about 30 mg/dL. The change in peak blood sugar in the subject can be determined by subtracting the peak blood sugar in the subject following energy consumption and the therapeutic composition from the peak blood sugar of the subject following energy consumption of the same composition without the therapeutic composition. The average change in peak blood sugar in the subject can be found by averaging the changes in peak blood sugar for a collection of subjects.

In some embodiments, a method of treating a metabolic disorder is provided. In some embodiments, the metabolic disorder is type-2 diabetes. In some embodiments, the method comprises the consumption or administration of a therapeutic composition as described herein to a subject in need thereof. In some embodiments, the subject uses a glucose monitor. In some embodiments, the subject uses information from the glucose monitor to know when to consume/administer the therapeutic composition. In some embodiments, the subject is obese. In some embodiments, the subject is prediabetic. In some embodiments, the subject is insulin resistant. In some embodiments, the subject has type-2 diabetes. In some embodiments, the subject has a disease or condition selected from the group consisting of obesity, prediabetes, insulin resistance, or type-2 diabetes. In some embodiments, the subject is obese and has type-2 diabetes. In some embodiments, the subject is undergoing additional treatment for the metabolic disorder. In some embodiments, the additional treatment comprises administration of metformin. In some embodiments, the additional treatment comprises insulin. In some embodiments, the additional treatment comprises one or more agents selected from the group consisting of metformin, insulin, alpha-glucosidase inhibitors, biguanides, dopamine agonists, dipeptidyl peptidase-4 (DPP-4) inhibitors, incretin mimetics, meglitinides, sodium-glucose transporter (SGLT)2 inhibitors, sulfonylureas, and thiazolidinediones.

In some embodiments, a method of increasing satiety in a subject is provided. In some embodiments, the method comprises consuming/administering a therapeutic composition as described herein. In some embodiments, the method is used by a subject who is obese. In some embodiments, the method is used for the purposes of weight loss. In some embodiments, the method is used by a subject who is prediabetic. In some embodiments, the method is used by a subject who is insulin resistant. In some embodiments, the method is used by a subject who has type-2 diabetes. In some embodiments, the method is used by a subject who has a disease or condition selected from the group consisting of prediabetes, insulin resistance, or type-2 diabetes. In some embodiments, the subject is obese and has one or more diseases or conditions selected from the group consisting of prediabetes, insulin resistance, type-2 diabetes, and insulin dependence. In some embodiments, the subject has one or more conditions selected from the group consisting of abdominal obesity, high blood pressure, high blood sugar, high serum triglycerides, and low serum high-density lipoprotein (HDL). In some embodiments, the subject is 40 years old or older. In some embodiments, the subject is undergoing treatment comprising administration of metformin. In some embodiments, the subject is undergoing treatment comprising administration of insulin.

Also provided herein is a method of treating a disease or disorder in which a microdose can help manage the disease or disorder and/or improve the therapeutic outcome of an existing pharmaceutical agent, wherein the method comprises administering to a subject in need thereof a therapeutic composition as provided herein.

In one variation, provided is a method of treating a metabolic disorder comprising administering to a subject in need thereof a therapeutic composition provided herein. In some embodiments, the metabolic disorder is selected from the group consisting of prediabetes, insulin resistance, type-2 diabetes, or insulin dependence. In some variations, the therapeutic composition for administration in such a method comprises a pharmaceutical agent for the treatment of such metabolic disorder, such as metformin. In some variations, the therapeutic composition for administration in such a method comprises both a pharmaceutical agent for the treatment of such metabolic disorder and a micronutrient such as fiber. In some variations, the therapeutic composition for administration in such a method comprises a pharmaceutical agent for the treatment of such metabolic disorder, such as metformin, and any one or more of a fiber, ergocalciferol, an incretin, an incretin mimetic, a sulfonylurea, a thiazolidinedione, or a gliptin.

In some embodiments, a method for improving efficacy of a pharmaceutical is provided. In some embodiments, a method of administering a pharmaceutical with a suitable number of calories is provided. In some embodiments, the method comprises consuming/administering a therapeutic composition as described herein. In some embodiments, the pharmaceutical is metformin. In some embodiments, the pharmaceutical is selected from the group consisting of metformin, an incretin, an incretin mimetic, a sulfonylurea, a thiazolidinedione, a gliptin, or any combination of the foregoing. In some embodiments, the pharmaceutical is insulin. In some embodiments, the pharmaceutical is selected from the group consisting of insulin, metformin, an incretin, an incretin mimetic, a sulfonylurea, a thiazolidinedione, a gliptin, or any combination of the foregoing. In some embodiments, the pharmaceutical is administered simultaneously with the therapeutic composition. In some embodiments, the pharmaceutical is administered after a period of time following consumption/administration of the therapeutic composition. In some embodiments, the period of time is 5, 10, 15, 20, 30, 45, or 60 minutes. In some embodiments wherein more than one pharmaceutical is administered, each pharmaceutical may be administered at a different time relative to the therapeutic composition.

Also provided herein are compositions for use in any of the methods detailed herein. Also provided are methods of preparing a medicament for use in any of the methods detailed herein.

The therapeutic compositions may be useful in conjunction with a pharmaceutical to enhance therapeutic outcomes by improving safety, efficacy, and/or subject compliance. In some embodiments, the therapeutic composition enhances the therapeutic effects of a treatment for a disease or condition. In some embodiments, the treatment is a pharmaceutical administered to a subject in need thereof. In some embodiments, the therapeutic composition is administered at the same time as the treatment. In some embodiments, the therapeutic composition is administered at a different time than the treatment. In some embodiments, the therapeutic composition comprises the pharmaceutical. In some embodiments, the therapeutic composition is administered to a subject in need thereof who is undergoing treatment for a disease or condition. In some embodiments, the therapeutic composition has an improved safety profile compared to a pharmaceutical treatment for the same disease or disorder.

In some embodiments of any of the preceding methods, the subject has a metabolic disorder. In some embodiments, the subject has a disease or condition selected from the group consisting of prediabetes, insulin resistance, type-2 diabetes, or insulin dependence. In some embodiments, the subject is obese. In some embodiments, the subject is obese and has one or more diseases or conditions selected from the group consisting of prediabetes, insulin resistance, type-2 diabetes, and insulin dependence. In some embodiments, the subject has one or more conditions selected from the group consisting of abdominal obesity, high blood pressure, high blood sugar, high serum triglycerides, and low serum high-density lipoprotein (HDL). In some embodiments, the subject is 40 years old or older. In some embodiments, the therapeutic composition comprises metformin. In some embodiments, the therapeutic composition comprises fiber. In some embodiments, the therapeutic composition comprises ergocalciferol. In some embodiments, the therapeutic composition comprises an incretin, an incretin mimetic, a sulfonylurea, a thiazolidinedione, or a gliptin.

In some embodiments of any of the above methods, the subject has developed or is at risk of developing pancreatitis. In some embodiments, the therapeutic composition is administered to a subject as a replacement for one or more incretin mimetic pharmaceuticals. In some embodiments, the subject is contraindicated for incretin mimetics. In some embodiments, the subject is at risk of developing hypoglycemia. In some embodiments, the subject is at risk of developing hypoglycemia if treated with one or more incretin mimetic pharmaceuticals.

In some embodiments, a method of assessing risk of hypoglycemia in a subject is provided, the method comprising:
a) administering a therapeutic composition, such as any of those described herein, to a subject;
b) measuring the blood glucose level of the subject over a 2-hour period;
c) determining whether or not the blood glucose level falls below 70 mg/mL at any point during the 2-hour period; and
d) determining that the subject has either a high or low risk of hypoglycemia, wherein the subject has a high risk of hypoglycemia if the blood glucose level falls below 70 mg/mL at any point during the 2-hour period and a low risk of hypoglycemia if the blood glucose level does not fall below 70 mg/mL at any point during the 2-hour period.
   In some embodiments, a patient determined to be at high risk of hypoglycemia using the above method is also determined to be a high-risk candidate for therapy comprising incretin mimetic drugs. In some embodiments, the patient is not a candidate for therapy comprising incretin mimetic drugs.

### Kits

In some embodiments, a kit comprising a therapeutic composition as described herein is provided. In some embodiments, the kit comprises a therapeutic composition as described herein and appropriate packaging. In some embodiments, the kit comprises a therapeutic composition as described herein and a device for administering the therapeutic composition. In some embodiments, the kit comprises a therapeutic composition as described herein and instructions for proper use of the therapeutic composition for treating a disease, improving the efficacy of a pharmaceutical, controlling, or attenuating glycemic response, preventing hyperglycemia, increasing satiety, or some combination of the above uses, as appropriate.

### EXAMPLES

### Example 1: Oral Glucose Tolerance Test in Healthy Adults

An oral glucose tolerance test (OGTT) was performed on 20 healthy adults in three separate iterations. The OGTT protocol was used to establish the baseline response to a glucose tolerance test beverage, the baseline response to an exemplary therapeutic composition, and the treatment response to administration of the exemplary therapeutic composition followed by the glucose tolerance test beverage. The test was administered by giving participants one of the following:
a) a 10 oz. glucose tolerance test beverage comprising 75 mg glucose (Trutol™ beverage or CHO);
b) a 10 oz. therapeutic composition consisting of 30 g extra virgin olive oil or medium-chain triglyceride (MCT) oil (randomly selected for each participant), and 7.5 g of collagen protein in water (fat/protein composition or FATPRO); or
c) the therapeutic composition of (b) followed by the beverage of (a) after 30 minutes (therapeutic combination or COMBO).

Fasting blood glucose was determined, and once each beverage condition was consumed, additional blood glucose samples (finger sticks) were taken at 15, 30, 60, and 120 min. Data were analyzed using a mixed factorial repeated measures ANOVA (analysis of variance). The software SPSS v. 24 was used for statistical analysis. Effect size (ES) was determined by calculating Cohen's d (for groups with equal sample size) and Hedge's g (for groups with unequal sample size). In general, effect size (meaningfulness of a difference) is small when ES equals 0.2, medium when ES equals 0.5, and large when ES equals 0.8. Data were analyzed comparing beverages based on sex, BMI, lipid source, and all factors combined.

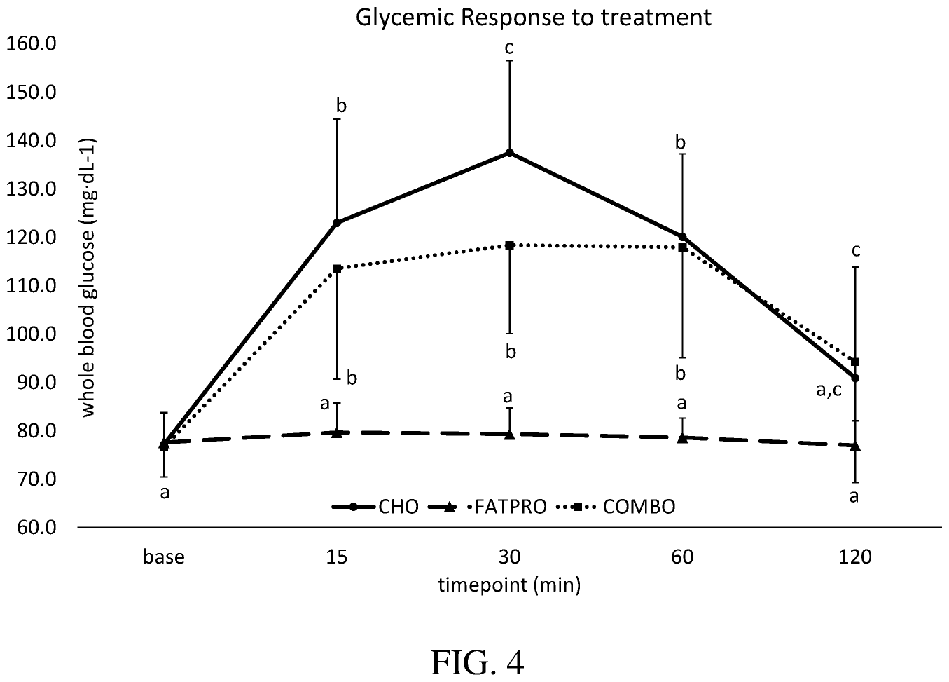

The results (see FIG. 4 to FIG. 11) demonstrate that the composition was effective at limiting the glycemic response in healthy participants.

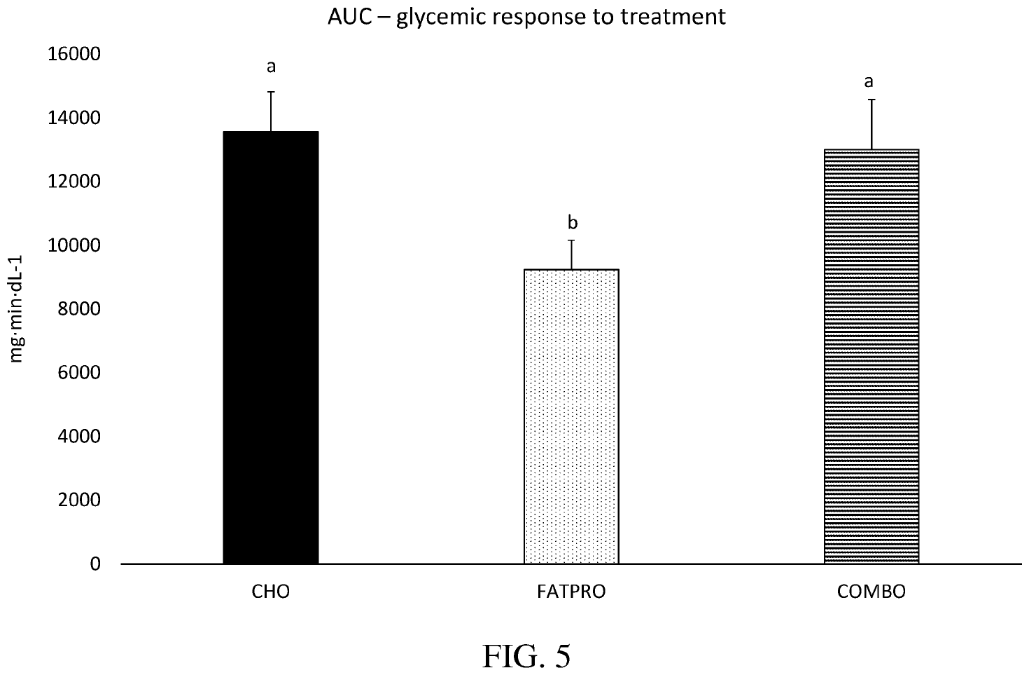

FIG. 4 and FIG. 5 show the data for all 20 participants combined. Different letters indicate significant difference at p<0.001. The AUC for Trutol™ vs. therapeutic combination (FIG. 5) was similar, but peak blood glucose was significantly lowered in participants when the therapeutic combination was consumed compared to when the Trutol™ beverage was consumed alone (see timepoint at 30 mins, FIG. 4).

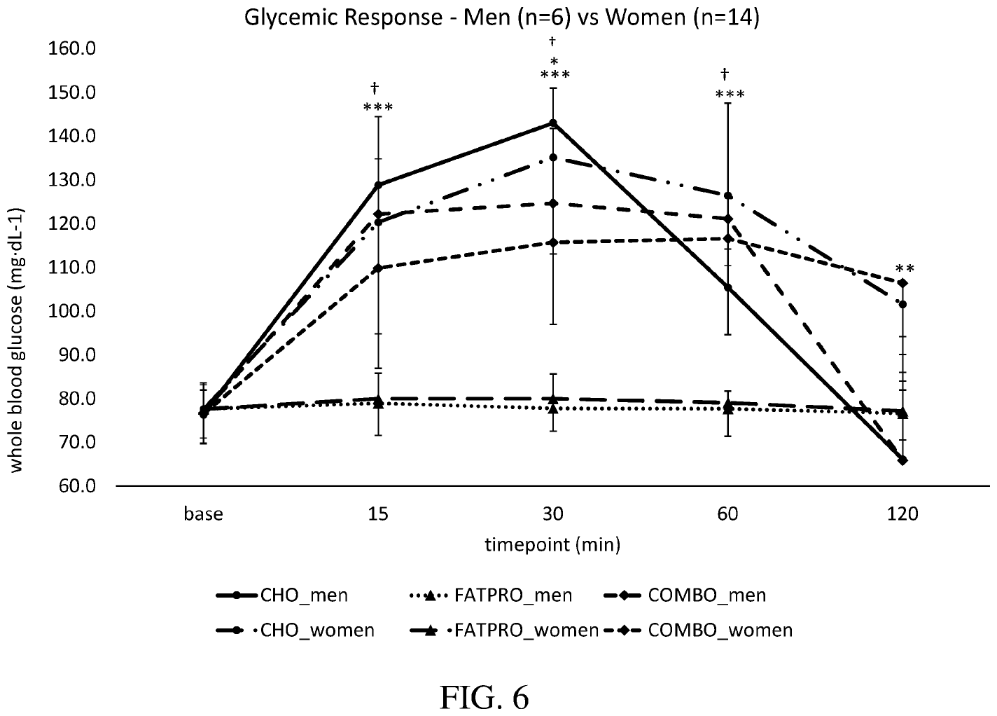

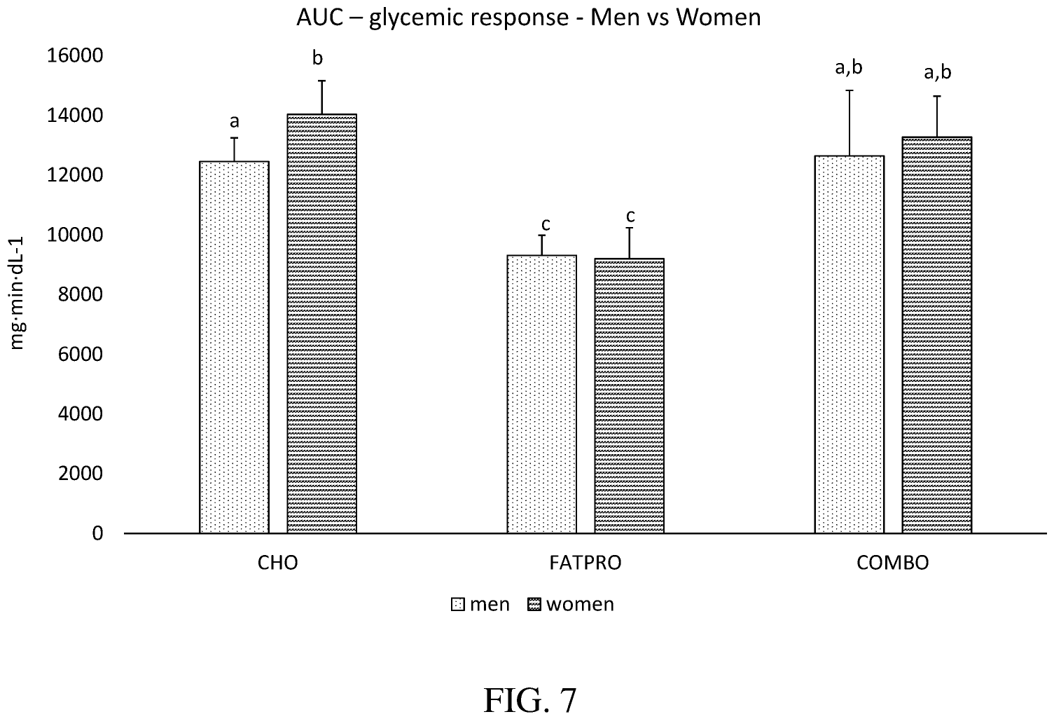

FIG. 6 and FIG. 7 show the data separated for men and women. Different letters indicate significant differences. For FIG. 6, p=0.006 between men and women who consumed Trutol™; p<0.001 between beverages regardless of sex. For FIG. 7, *p=0.01 in women between Trutol™ consumption and therapeutic composition consumption at 30 min; **p<0.001 between consumption of Trutol™ and the therapeutic combination in men and women at 120 min; ***p<0.001 between consumption of the fat/protein composition and either Trutol™ or the therapeutic composition at the indicated timepoints; †p<0.001 from baseline. The glycemic response was significantly lower in women following consumption of the therapeutic combination than following consumption of Trutol™ (ES: d=0.95; see FIG. 6). The glycemic response in men was not significantly lower between consumption of the therapeutic composition and consumption of Trutol™ (p=0.08), but the meaningfulness of the difference was high (ES: d=1.37; see FIG. 6). Glycemic response was significantly lower in men than in women who consumed Trutol™ (ES: g=2.18) and the therapeutic combination (ES: g=2.36) at 120 minutes (see FIG. 6). Blood glucose AUC was generally higher for women than for men within beverage groups (ES: g=1.51; see FIG. 7).

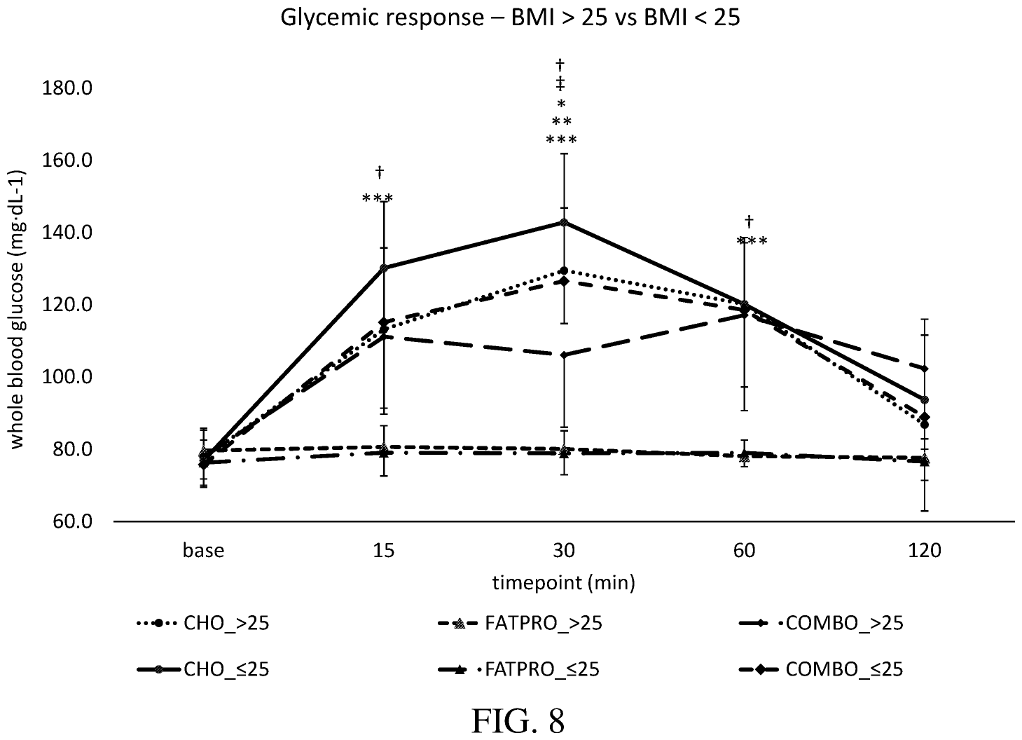

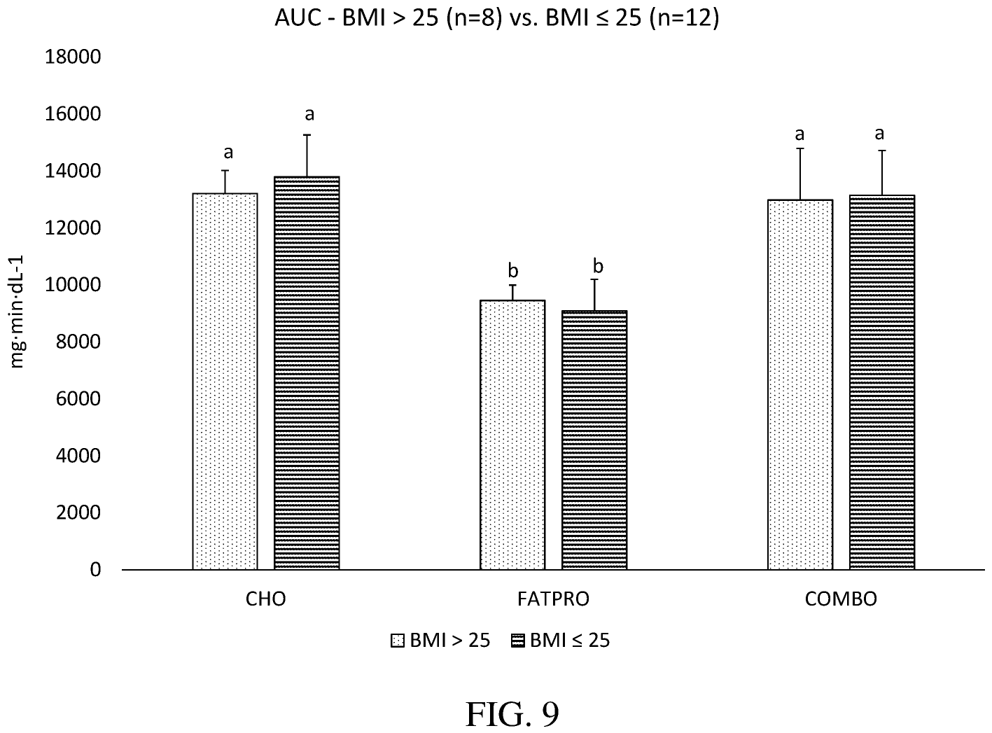

FIG. 8 and FIG. 9 show the data separated for participants with a BMI (Body Mass Index) higher than 25 and a BMI lower than or equal to 25. For FIG. 8, different letters indicate significant difference at p<0.001. For FIG. 9, *p=0.01 between consumption of Trutol™ and consumption of the therapeutic combination in participants with a BMI ≥25 at 30 min; **p=0.05 between consumption of Trutol™ and consumption of the therapeutic combination in participants with BMI ≤25 at 30 min; ***p<0.001 between consumption of the fat/protein composition and either Trutol™ or the therapeutic composition at indicated timepoints; †p<0.001 from baseline; ‡p=0.01 between participants with BMI >25 and ≤25 in COMBO at 30 min. Glycemic response was significantly less at 30 minutes for participants who consumed the therapeutic combination compared to those that consumed the Trutol™ beverage for the BMI >25 group (ES: d=1.25) and the BMI ≤25 group (ES: d=1.02; see FIG. 8). Glycemic response was significantly less at 30 minutes for participants with BMI >25 than participants with BMI ≤25 (ES: 1.32; see FIG. 8) and the difference was highly meaningful.

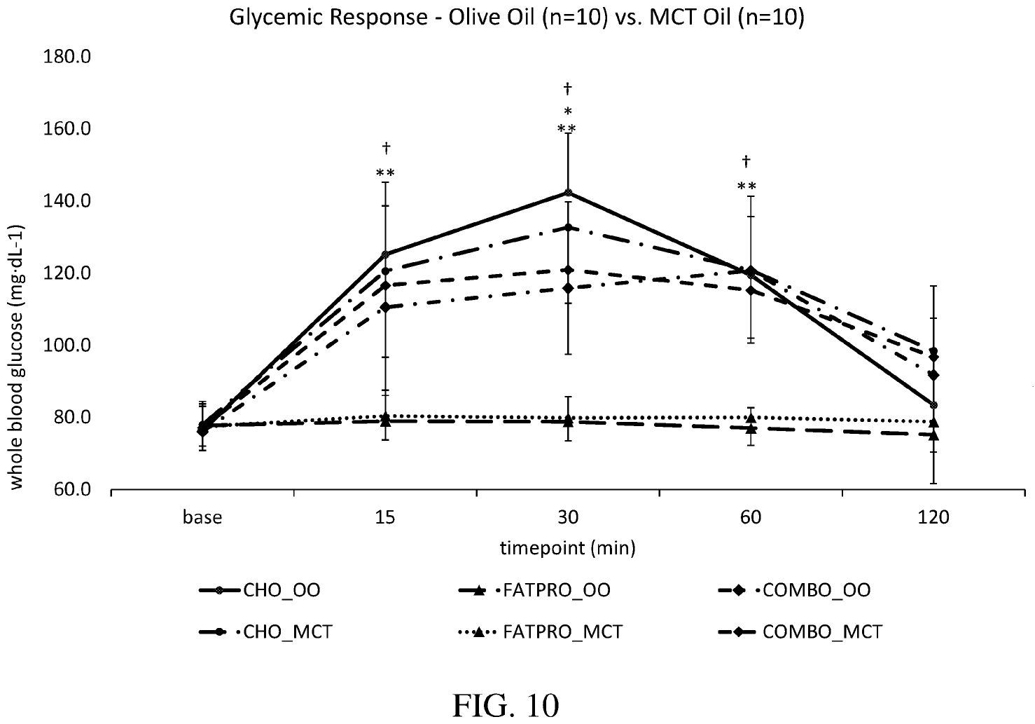

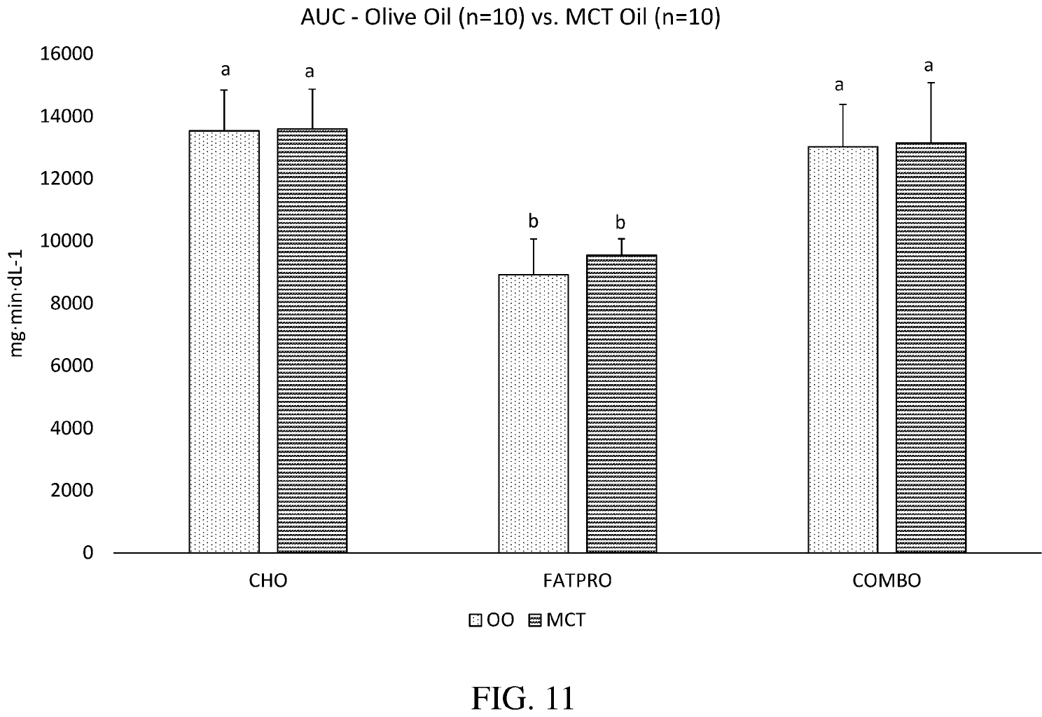

FIG. 10 and FIG. 11 show the data separated for participants using the fat/protein composition or therapeutic combination with olive oil and the fat/protein composition or therapeutic combination with medium-chain triglyceride (MCT) oil. For FIG. 10, different letters indicate significant difference at p<0.001. For FIG. 11, *p=0.006 between consumption of the therapeutic combination than following consumption of Trutol™ in participants consuming olive oil at 30 min; **p<0.001 between consumption of the fat/protein composition and consumption of the therapeutic combination and consumption of Trutol™ at indicated timepoint; †p<0.001 from baseline. FIG. 10 shows that the glycemic response was lowered more effectively (compared to the Trutol™ beverage) for participants who consumed the therapeutic combination with olive oil compared to those that consumed the therapeutic combination with MCT oil.

### Example 2: Compositions

The therapeutic compositions in Table 1 are examples of therapeutic compositions described above. See FIG. 7 for examples of the components that can be included in the therapeutic compositions.

TABLE 1
Examples of therapeutic compositions.

| Formula | | | |
|---|---|---|---|
| number | Fatty acid component | Amino acid component | Additional agent |
| 1 | about 2-4 g fatty acid | about 2-5 g amino acid | ergocalciferol |
| | | composition | |
| 2 | about 8-10 g fatty acid | about 10-12 g amino acid | ergocalciferol |
| | | composition | |
| 3 | about 2-4 g fatty acid | about 6-8 g amino acid | ergocalciferol |
| | | composition | |
| 4 | about 8-11 g fatty acid | about 20-22 g amino acid | ergocalciferol |
| | | composition | |
| 5 | about 28-32 g fatty acid | about 6-9 g amino acid | psyllium |
| | | composition | |
| 6 | about 8-11 g fatty acid | about 19-23 g amino acid | metformin |
| | | composition | (microencapsulated) |
| 7 | about 2-4 g fatty acid | about 6-8 g amino acid | caffeine |
| | | composition | |
| 8 | about 8-11 g fatty acid | about 19-23 g amino acid | caffeine |
| | | composition | |
| 9 | about 2-4 g fatty acid | about 6-8 g amino acid | epigallo-catechin gallate |
| | | composition | |
| 10 | about 8-11 g fatty acid | about 19-23 g amino acid | epigallo-catechin gallate |
| | | composition | |

The compositions in Table 2 are examples of unit dosage forms of the compositions described above.

TABLE 2
Examples of therapeutic compositions.

| Formula | | Fatty acid | | |
|---|---|---|---|---|
| number | Calories | component | Amino acid component | Additional agent |
| 1 | 41.4 | 3 g extra virgin olive | 3.6 g whey protein isolate or | 200 i.u. ergocalciferol |
| | | oil or MCT oil | collagen peptide | |
| 2 | 124 | 9 g extra virgin olive | 10.8 g whey protein isolate | 200 i.u. ergocalciferol |
| | | oil or MCT oil | or collagen peptide | |
| 3 | 56.8 | 3.2 g extra virgin | 7 g whey protein isolate or | 200 i.u. ergocalciferol |
| | | olive oil or MCT oil | collagen peptide | |
| 4 | 170.4 | 9.6 g extra virgin | 21 g whey protein isolate or | 200 i.u ergocalciferol |
| | | olive oil or MCT oil | collagen peptide | |
| 5 | 324 | 30 g extra virgin | 7.5 g whey protein isolate or | 6 g psyllium |
| | | olive oil or MCT oil | collagen peptide | |
| 6 | 170.4 | 9.6 g extra virgin | 21 g whey protein isolate or | 500 mg metformin |
| | | olive oil or MCT oil | collagen peptide | (microencapsulated) |
| 7 | 56.8 | 3.2 g extra virgin | 7 g whey protein isolate or | 100 mg caffeine |
| | | olive oil or MCT oil | collagen peptide | |
| 8 | 170.4 | 9.6 g extra virgin | 21 g whey protein isolate or | 100 mg caffeine |
| | | olive oil or MCT oil | collagen peptide | |
| 9 | 56.8 | 3.2 g extra virgin | 7 g whey protein isolate or | 100 mg epigallo- |
| | | olive oil or MCT oil | collagen peptide | catechin gallate |
| 10 | 170.4 | 9.6 g extra virgin | 21 g whey protein isolate or | 100 mg epigallo- |
| | | olive oil or MCT oil | collagen peptide | catechin gallate |

We claim:

1. A therapeutic composition comprising:
(a) a fatty acid; and
(b) an amino acid composition,
wherein the therapeutic composition has a total number of calories, and
wherein (a) and (b) taken together comprise a number of calories accounting for at least 50% of the total number of calories in the therapeutic composition, and
wherein the therapeutic composition has a caloric density of at least 100 calories per ounce.

2. The therapeutic composition of claim 1, wherein the fatty acid comprises an unsaturated fatty acid.

3. The therapeutic composition of claim 1 or 2, wherein the fatty acid comprises a medium-chain triglyceride.

4. The therapeutic composition of any one of claims 1 to 3, wherein the fatty acid comprises a vegetable oil.

5. The therapeutic composition of claim 4, wherein the vegetable oil is selected from the group consisting of olive oil, canola oil, avocado oil, coconut oil, peanut oil, walnut oil, sesame oil, soybean oil, almond oil, flaxseed oil, and sunflower oil.

6. The therapeutic composition of claim 5, wherein the vegetable oil is olive oil.

7. The therapeutic composition of claim 6, wherein the olive oil is extra virgin olive oil.

8. The therapeutic composition of claim 1, wherein the fatty acid comprises two or more fatty acids.

9. The therapeutic composition of claim 8, wherein the two or more fatty acids comprise olive oil and a medium-chain triglyceride oil.

10. The therapeutic composition of any one of claims 1 to 9, wherein the amino acid composition comprises a straight-chain amino acid or a branched-chain amino acid.

11. The therapeutic composition of any one of claims 1 to 9, wherein the amino acid composition comprises whey protein, whey protein isolate, collagen protein, collagen peptide, plant-based protein or insect-based protein, or a fragment of any of the foregoing.

12. The therapeutic composition of any one of claims 1 to 9, wherein the amino acid composition comprises pea protein, hemp protein or cricket protein, or a fragment of any of the foregoing.

13. The therapeutic composition of any one of claims 1 to 12, further comprising a microparticle having a size of between about 500 microns across at its widest point and about 3000 microns across at its widest point.

14. The therapeutic composition of claim 13, wherein the microparticle comprises the amino acid composition and is suspended in a carrier.

15. The therapeutic composition of claim 14, wherein the carrier comprises the fatty acid.

16. The therapeutic composition of any one of claims 1 to 15, further comprising an additional agent.

17. The therapeutic composition of claim 16, wherein the additional agent is dissolved or suspended in the therapeutic composition.

18. The therapeutic composition of any one of claims 13 to 15, wherein the microparticle comprises an additional agent.

19. The therapeutic composition of any one of claims 16 to 18, wherein the additional agent is a micronutrient, a probiotic, a stimulant, or a pharmaceutical agent.

20. The therapeutic composition of any one of claims 16 to 19, wherein the additional agent is selected from the group consisting of ergocalciferol, cholecalciferol, calcium, pectin, psyllium, caffeine, metformin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone.

21. The therapeutic composition of any one of claims 1 to 20, further comprising a flavor-masking agent.

22. The therapeutic composition of claim 21, wherein the flavor-masking agent is selected from the group consisting of limonene, methyl salicylate, diacetyl, acetylpropionyl, acetoin, peppermint oil, and cinnamaldehyde.

23. The therapeutic composition of claim 22, wherein the flavor-masking agent is cinnamaldehyde.

24. The therapeutic composition of any one of claims 1 to 23, wherein the ratio of the (a) fatty acid to the (b) amino acid composition is between 20:1 and 1:20 by weight.

25. The therapeutic composition of claim 24, wherein the ratio is between 20:1 and 1:1 by weight.

26. The therapeutic composition of claim 25, wherein the ratio is between 20:1 and 5:1 by weight.

27. The therapeutic composition of any one of claims 1 to 26, wherein the therapeutic composition has a caloric density of about 100 to about 300 calories per ounce.

28. The therapeutic composition of any one of claims 1 to 27, wherein the therapeutic composition is packaged as a unit dose.

29. The therapeutic composition of claim 28, wherein the unit dose comprises no more than 1.5 ounces of the therapeutic composition.

30. The therapeutic composition of claim 28, wherein the unit dose is formulated as a gelatin droplet.

31. The therapeutic composition of claim 28, wherein the unit dose is formulated as a liquid-filled capsule or lozenge.

32. The therapeutic composition of claim 28, wherein the unit dose is formulated as a suspension packaged in a suitable container.

33. The therapeutic composition of claim 32, wherein the suspension contains 40 to 350 calories and has a volume of 0.2 to 1.0 ounces.

34. The therapeutic composition of any one of claims 1 to 33, wherein the therapeutic composition is free from a natural or an artificial sweetener.

35. The therapeutic composition of any one of claims 1 to 34, further comprising a probiotic.

36. The therapeutic composition of claim 35, wherein the probiotic is one or more colonies of microbes.

37. The therapeutic composition of any one of claims 1 to 36, further comprising a fullerene.

38. The therapeutic composition of any one of claims 1 to 37, further comprising a taste receptor agonist with an enteric coating.

39. The therapeutic composition of any one of claims 1 to 38, wherein the therapeutic composition comprises no more than 5% carbohydrates by weight.

40. A method of stimulating the release of one or more incretins in a subject, comprising administering to the subject a therapeutic composition according to any one of claims 1 to 39, wherein the therapeutic composition is administered to the subject not more than one hour prior to routine energy consumption.

41. A method of attenuating glycemic response in a subject in need thereof, comprising administering to the subject a therapeutic composition according to any one of claims 1 to 39, wherein the therapeutic composition is administered to the subject not more than one hour prior to routine energy consumption.

42. A method of increasing satiety in a subject, comprising administering to the subject a therapeutic composition according to any one of claims 1 to 39, wherein the therapeutic composition is administered to the subject not more than one hour prior to routine energy consumption.

43. The method of any one of claims 40 to 42, wherein the therapeutic composition is administered to the subject not more than 5, 10, 15, or 30 minutes prior to routine energy consumption.

44. A method of providing a meal replacement to a subject, comprising administering the therapeutic composition of any one of claims 1 to 39 in place of routine energy consumption.

45. The method of any one of claims 40 to 44, wherein the subject is obese.

46. The method of any one of claims 40 to 45, wherein the subject has a metabolic disorder.

47. The method of claim 46, wherein the metabolic disorder is prediabetes, insulin resistance, or type-2 diabetes.

48. The method of claim 46 or 47, wherein the subject is undergoing treatment for the metabolic disorder.

49. The method of claim 48, wherein the subject is concurrently being treated with a pharmaceutical agent selected from the group consisting of metformin, insulin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone.

50. A method of improving efficacy of a pharmaceutical in a subject, comprising administering to a subject receiving treatment with the pharmaceutical the therapeutic composition of any one of claims 1 to 39 not more than one hour prior to routine energy consumption.

51. The method of claim 50, wherein the pharmaceutical is selected from the group consisting of metformin, insulin, acarbose, miglitol, bromocriptine, alogliptin, albiglutide, dulaglutide, exenatide, nateglinide, repaglinide, dapagliflozin, canagliflozin, empagliflozin, glimepiride, gliclazide, rosiglitazone, and pioglitazone.

52. A method of treating a disease or disorder in a subject in need thereof, comprising administering to the subject the therapeutic composition of any one of claims 1 to 39 not more than one hour prior to routine energy consumption, wherein the disease or disorder is type-2 diabetes.

53. The method of claim 52, wherein the therapeutic composition is administered to the subject not more than 5, 10, 15, or 30 minutes prior to routine energy consumption.

54. A method of assessing risk of hypoglycemia in a subject is provided, the method comprising:
a) administering the therapeutic composition of any one of claims 1 to 39 to a subject;
b) measuring a blood glucose level of the subject over a 2-hour period;
c) determining whether or not the blood glucose level falls below 70 mg/mL at any point during the 2-hour period; and
d) determining that the subject has either a high or low risk of hypoglycemia, wherein the subject has a high risk of hypoglycemia if the blood glucose level falls below 70 mg/mL at any point during the 2-hour period and a low risk of hypoglycemia if the blood glucose level does not fall below 70 mg/mL at any point during the 2-hour period.

55. A kit comprising the composition of any one of claims 1 to 39 and suitable packaging for the therapeutic composition.
